(19) United States
(12) Patent Application Publication
(10) Pub. No.: US 2026/0036522 A1
(43) Pub. Date: Feb. 5, 2026

(54) WATER TEST SYSTEM

(71) Applicant: CoralVue, Slidell, LA (US)

(72) Inventors: David D'Aquin, Slidell, LA (US); Don Edvalson, Slidell, LA (US)

(21) Appl. No.: 18/792,388

(22) Filed: Aug. 1, 2024

(51) Int. Cl. G01N 21/78; G01N 21/03; G01N 33/18; G01N 35/00; G01N 35/10

(52) U.S. Cl. CPC G01N 21/78; G01N 21/0303; G01N 33/18; G01N 35/1002; G01N 2035/00534

(57) ABSTRACT

Disclosed herein are test assemblies for use with an aquatic system such as an aquarium. The test assemblies can include an optical test environment that includes a sample isolation space, such as a cuvette, an optical emitter, and an optical receptor. In some examples, the optical test environment also includes a reflective surface. The test assemblies can also include a manifold connected to the optical test environment and configured to introduce controlled liquid volumes into the test environment. In some examples, the manifold may also be equipped with one or more air or liquid pumps configured to flush fluids from the manifold. In some examples, the test assembly may include a computerized controller. In some examples, the test assembly can include a homogenization device, such as a magnetic stirrer.

### CROSS-REFERENCE TO RELATED APPLICATIONS

This application claims benefit of U.S. Provisional Patent Application No. 63/636,444, filed on Apr. 19, 2024 which is hereby incorporated by reference.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND

During the use of an aquatic system such as an aquarium, aquaculture container, breeding system or terrarium; it is expected that various impurities, including biological and chemical material buildup, will accumulate in the water in the system. It is therefore necessary to periodically test and monitor the system water to determine both which impurities may have accumulated, and in what concentrations such impurities have accumulated in the aquarium water. In addition to impurities, it is also necessary to test the levels of certain desirable chemicals in the water.

One test method involves reacting a sample of water with one or more chemical reagents which exhibit a visual and/or optical reaction when exposed to one or more expected chemicals in a water sample. This allows for subsequent optical detection of such chemicals. However, existing optical test systems may be limited in sensitivity, may position sensitive optical equipment in locations where water splash and/or leakage may cause damage, and may make the cleaning and maintenance of the optical test system difficult.

There is, therefore, a need for improved optical test systems to conduct chemical testing of aquarium water samples, and devices for accomplishing the same.

### SUMMARY

Disclosed herein are liquid test systems which may be suitable for testing for chemical content in water, and particular, the sorts of chemicals expected in aquatic system water. Also disclosed herein are pumping assemblies for use in the liquid test systems disclosed herein. Also disclosed herein are cuvette assemblies for use in the liquid test systems disclosed herein.

Certain examples concern a liquid test system. The liquid test system comprises a cuvette configured to contain a water sample; a reflective surface positioned on a first side of the cuvette; a light emitter positioned on a second side of the cuvette opposite to the reflective surface; and a light sensor positioned on a second side of the cuvette opposite to the reflective surface. The light emitter is configured to transmit a light through the cuvette and the water sample. The reflective surface is configured to receive the light transmitted through the cuvette and the water sample and reflect the light through the cuvette and the water sample. The light sensor is configured to receive the reflected light and to generate a sensor signal based on the reflected light.

Certain examples concern a liquid test system. The liquid test system comprises a cuvette configured to contain a water sample; a light emitter positioned adjacent to the cuvette; a light sensor positioned adjacent to the cuvette and alongside the light emitter; and a pumping assembly. The pumping assembly includes a manifold, a liquid pump in communication with the manifold, an airtight valve in communication with the manifold, and one or more liquid containers in communication with the manifold. The light emitter is configured to transmit a light through the cuvette and the water sample. The light sensor is configured to receive the transmitted light and to generate a sensor signal based on the received light. The liquid pump is disposed between the manifold and the cuvette, and configured to introduce liquids from the manifold to the cuvette. The airtight valve is configured to introduce an airflow into the manifold, such that a liquid content of the manifold is expelled from the manifold.

Certain examples concern a water test method, the method. The water test method comprises introducing a liquid sample to a cuvette through a conduit; introducing a reagent to the liquid sample; transmitting light a first time through the liquid sample such that the light reflects off a reflective surface; and transmitting the light through the liquid sample a second time such that the light is received by a light sensor.

Numerous objects, features and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1A is a side view schematic of an optical water testing system according to one aspect of the present disclosure.

FIG. 1B is a top view schematic of the optical water testing system of FIG. 1A.

FIG. 2 is a schematic of a liquid flow system according to one aspect of the present disclosure.

FIG. 3 is a side schematic view of a cuvette assembly according to one aspect of the present disclosure.

FIG. 4 is a side schematic view of a test assembly, including a test system, a cuvette assembly, and a liquid flow system according to one aspect of the present disclosure.

FIG. 5 is a schematic diagram of a controller for use with the water test systems of the present disclosure.

FIG. 6A is a perspective view of a test assembly according to one aspect of the present disclosure.

FIG. 6B is a cutaway view of the test assembly of FIG. of 6A.

FIG. 6C is a cutaway view of the test assembly of FIG. 6A with the cuvette removed.

FIG. 7 is a flow chart illustrating the steps of a method for preparing a test sample using a test assembly according to one aspect of the present disclosure.

### DETAILED DESCRIPTION

### General Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

Unless explained otherwise, all technical and scientific terms used herein have the same meaning as commonly understood to one of ordinary skill in the art to which this disclosure belongs. Although methods and devices similar or equivalent to those described herein can be used in the practice or testing of the present disclosure, suitable methods and devices are described below. The devices, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

### Introduction to the Disclosed Technology

To facilitate testing of liquid samples that may include various chemicals which can change in level in the system water over time, it may be useful to isolate a sample of water in a testing environment under which it can be subjected to one or more chemical or physical tests.

One such method involves isolating a liquid sample in a cuvette with one or more chemical reagents that will react to various chemicals being tested for. In such cases, the reaction of the chemical with the reagents can be detected with one or more sensors, such as optical sensors, which can detect changes in opacity, color, transmissivity, or other optical properties of the liquid sample which may change following a reaction with a chemical reagent.

However, the process of administering reagents to an isolated liquid sample for testing raises several important technical challenges. For example, an isolated test environment ideally requires only small test samples of the liquid (such as aquarium water) to be tested. In turn, this may require that only limited quantities of a reagent are added to the isolated test environment, or that any measured reaction must be normalized to account for differing amounts of reagent added. Accordingly, administering reagents in precisely controlled quantities may become important for ensuring the accuracy of any test. Because these reagents are typically administered to the isolated test environment through one or more pipes, conduits, or hoses, however, a portion of the administered reagent may become entrapped or otherwise left behind in the pipes, conduits, and/or hoses through which it must travel to the isolated test environment, and accordingly, the quantity of reagent that reaches the test environment may be less than that added to the test system by an unknown amount.

Additionally, there may be precision limits to the test, particularly an optical test, based on the limited volume of mixed reagent and water through which light passes and with which light interacts before being measured.

Furthermore, care must be taken in administering such tests that any measurement equipment, particularly electronic equipment, is kept isolated from the liquid sample being tested.

Disclosed herein are example liquid test systems which reduce or resolve these challenges, and which furthermore offer other advantages disclosed in greater detail below.

### Aspects of the Disclosed Technology

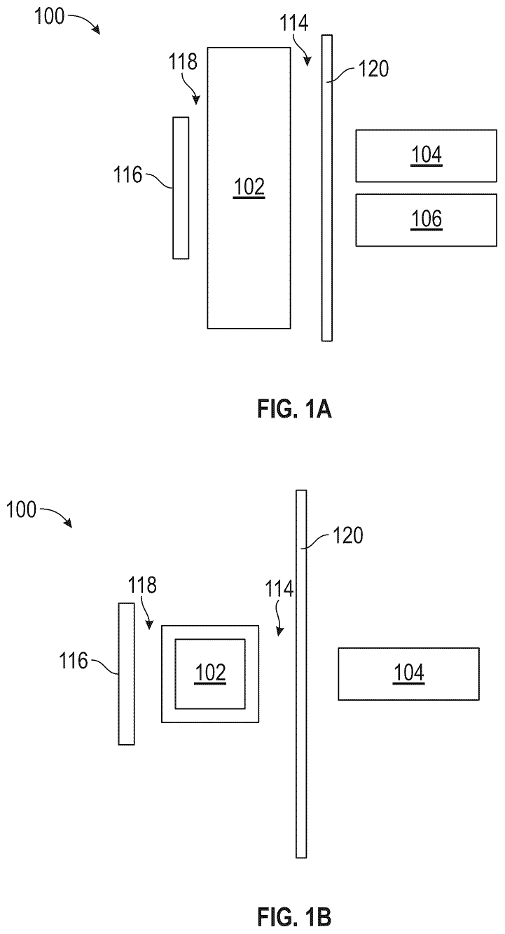

Disclosed herein are examples of a liquid test system, such as the liquid test system 100 shown in FIGS. 1A and 1B. According to one aspect of the present disclosure, the liquid test system 100 includes a cuvette 102 (sometimes called a tank 102), a light emitter 104, and a light sensor 106.

The cuvette 102 contains a sample of liquid, such as water from an aquatic system to be tested. According to some aspects of the present disclosure, the water in the cuvette 102 can be automatically channeled, directed, and/or pumped into the cuvette 102 from the tank by a plumbing or pumping assembly, such as those disclosed in greater detail below. The plumbing or pumping assembly can comprise one or more pumps or valves, a water conduit and/or tubing, and a controller, such as the controllers disclosed in greater detail below, which directs the pumps, valves, and other controllable components of the plumbing or pumping assembly to direct a sample of the test water to the cuvette 102, via one or more signals. The plumbing assembly can also be used to deliver one or more chemicals and/or reagents to the cuvette 102, as further discussed herein.

According to one aspect of the present disclosure, the light emitter 104 and the light sensor 106 are disposed to a first side 114 of the cuvette 102, as shown in FIGS. 1A and 1B. In some examples, the light emitter 104 and the light sensor 106 may be arranged side by side, and in other examples, the light emitter 104 and the light sensor 106 can be spaced apart either laterally or vertically.

According to one aspect of the present disclosure, the liquid test system 100 also includes a reflective surface 116 disposed to a second side 118 of the cuvette 102 opposite the first side 114 of the cuvette 102, as shown in FIGS. 1A and 1B. In this arrangement, the light emitter 104 emits light, which passes through the cuvette 102 and any water, chemicals, and/or reagents contained therein. The light emitted by the light emitter 104 reflects off the reflective surface 116, and thus can be detected by the light sensor 106 positioned alongside the light emitter 104. Thus, a light can thereby be transmitted through the cuvette 102 and the water, chemicals, and/or reagents contained therein without the need to install electrical components, such as the light emitter 104 and the light sensor 106 ono opposing sides of the water tank. Advantageously, this configuration facilitates the removal of the cuvette 102 from an aquarium assembly, which is periodically required to clean the cuvette 102.

According to one aspect of the present disclosure, the liquid test system 100 also includes a transparent waterproof barrier 120 disposed between the light emitter 104 and the cuvette 102. The transparent waterproof barrier 120 transmits the light emitted by the light emitter 104 as well as the light reflected towards the light sensor 106 by the reflective surface 116, but prevents water, such as water that may overflow or spill from the cuvette 102 from reaching either the light emitter 104 or the light sensor 106. Furthermore, according to one aspect of the present disclosure, the transparent waterproof barrier 120 can also be impermeable to chemicals and/or reagents in liquid and gaseous form, which can further protect the light emitter 104 and the light sensor 106 from damage or degradation resulting from chemical exposure.

In a generalized aspect of the present disclosure, water testing can be performed on a water sample using the liquid test system 100 as disclosed herein according to the following procedure.

Water, such as a water sample from the aquatic system. 108 is admitted to the cuvette 102 through the plumbing assembly under the control of the controller 112. One or more chemical reagents are also added to the cuvette 102, which may react with one or more compounds, such as biological residue, cellular matter, chemicals or aquarium waste, etc. present in the water sample. On reacting, the one or more chemical reagents may change the color or opacity of the water sample.

Following exposure of the water sample to the one or more chemical reagents, light is emitted from the light emitter 104 and transmitted through the water sample. As will be appreciated by one of skill in the art, the optic characteristics of the water sample, including the color and opacity of the water sample, will influence what wavelengths of light in the visual spectrum will be transmitted through the cuvette 102, and also the intensity at which that light is transmitted.

The transmitted light is reflected by the reflective surface 116 and passes through the cuvette 102 and the water sample contained in the cuvette 102 a second time, before being detected by the light sensor 106. The light sensor 106 then measures one or more optical characteristics, such as the wavelength, visual spectrum profile, or intensity of the transmitted light. The measured one or more optical characteristics can then be compared against known and/or expected optical characteristics that result from positive and/or negative presence of any of the biological residue, cellular matter, chemicals, or aquarium waste, etc. present in the water sample.

Advantageous, because the light from the light emitter 104 passes through the cuvette 102 and the water sample in the cuvette 102 two times, any optical filtering and/or blocking effects of the water sample on the light occur twice. In some aspects of the present disclosure, this may allow for the detection of smaller concentrations of any of the biological residue, cellular matter, chemical contamination, or aquarium waste, etc. present in the water sample, because any changes in the optical properties of the water sample due to the reaction of the chemicals and/or reagents will be measured twice.

Also disclosed herein are example pumping assemblies for a liquid test system, such as the liquid test system 100 disclosed herein.

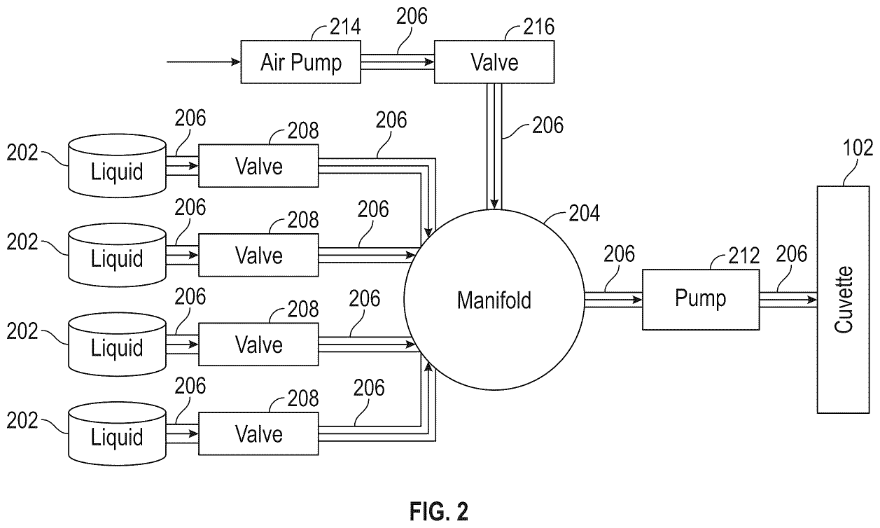

FIG. 2 shows a pumping assembly 200 for a liquid test system according to one aspect of the present disclosure. As shown in FIG. 2, the pumping assembly 200 can comprise liquid containers 202 (for example, liquid containers 202 associated with an aquarium) connected to a manifold 204 by one or more conduits 206 (sometimes called tubes 206). Thus, the manifold is in liquid communication with the liquid containers 202 through the one or more conduits 206.

The one or more conduits 206 can be regulated by one or more corresponding liquid valves 208. The liquid valves 208 can be moved between an open position, in which liquid, such as water, is permitted to pass through the valve, and a closed position, in which the liquid is prevented from passing through the valve. According to one aspect of the present disclosure, the number of liquid valves 208 can equal the number of conduits 206, such that each conduit is regulated by a corresponding valve 208. However, it will be appreciated that in other examples, the number of conduits 206 can differ from the number of valves, such that one or more of the conduits 206 are not governed by a valve 208, or one or more of the conduits 206 is governed by more than one valve 208. According to one aspect of the present disclosure, the one or more liquid valves 208 can be controlled (that is, moved between the open position and the closed position) in response to a signal from a controller, such as the controller 112 previously introduced in relation to the liquid test system 100 and shown in FIGS. 1A and 1B.

According to one aspect of the present disclosure, the manifold 204 can be in liquid communication with a liquid reservoir 210, which in some examples, may be the cuvette 102 previously introduced in relation to the liquid test system 100 and shown in FIGS. 1A and 1B.

According to one aspect of the present disclosure, the pumping assembly 200 can include a liquid pump 212 disposed between the manifold 204 and the liquid reservoir 210. In some examples, the liquid pump 212 is oriented to induce a liquid flow from the manifold 204 to the liquid reservoir 210 (that is, the liquid pump 212 can be downstream of the manifold 204 and upstream of the liquid reservoir 210). In some examples, the liquid pump 212 can be controlled by a controller, such as the controller 112, which can activate or deactivate the liquid pump 212 or increase and/or decrease the speed, and thus the flow rate, of the liquid pump 212.

In some examples, the liquid pump 212 can be directly connected to the manifold 204 and the liquid reservoir 210. In other examples, a length of conduit or tubing 206 can extend between the manifold 204 and the liquid reservoir 210 and the liquid pump 212 can be mounted in-line with the conduit 206.

The pumping assembly 200 also includes an air pump 214, which can also be in liquid communication with the manifold 204, as shown in FIG. 2. The air pump 214 can be separated from the manifold 204 by an airtight valve 216. The airtight valve 216 can be movable between an open position that allows air to pass from the air pump 214 into the manifold 204 and a closed position that prevents air from flowing through the airtight valve 216. The air pump 214 can be controlled by signals from a controller, such as the controller 112 previously introduced, which instruct the opening and closing of the airtight valve 216.

By activating the air pump 214 and opening the airtight valve 216 with the liquid valves 208 closed, liquid can be forced from the manifold 204 into the liquid reservoir 210. By activating the air pump 214 and opening the airtight valve 216 with the liquid valves 208 open, and with the liquid pump 212 closed or inactive, liquid can be forced from the manifold 204 and back into the liquid containers 202, or expelled from the manifold 204 to clear the manifolds of liquid. It will be appreciated that while, in some instances, the air pump 214 may not be needed to accomplish the emptying and/or flushing of the manifold 204, the air pump 214 allows a pressurized airflow to be provided, and that such pressurized airflows may advantageously be more effective at emptying or flushing the manifold 204, thereby removing various chemicals which can change in level in the system.

In this way, a pumping assembly 200 is provided that allows the various liquid-bearing components of the pumping assembly 200 to be easily flushed (that is, liquids and particularly residual liquids can be expelled from the pumping assembly), preventing the buildup of stagnant volumes of liquid within the pumping assembly 200.

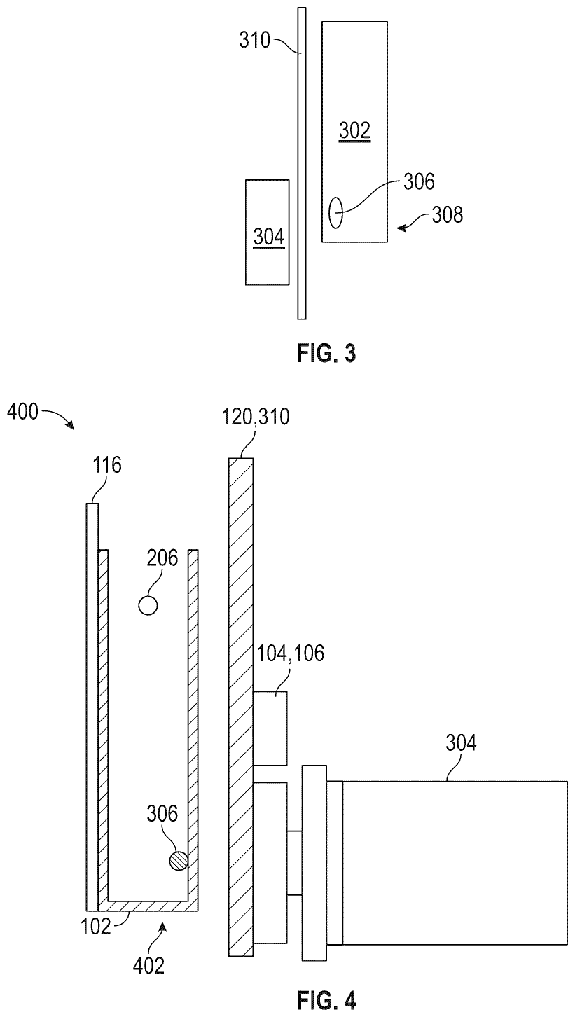

Also disclosed herein are example cuvette configurations, such as shown with respect to the cuvette assembly 300 of FIG. 3.

As shown in FIG. 3, the cuvette assembly 300 comprises a cuvette tank 302 and a magnetic motor 304 disposed adjacent to the cuvette tank 302. The cuvette tank 302 contains a magnetic stir bar 306, which is disposed towards a bottom end portion 308 of the cuvette tank 302. The magnetic motor 304 magnetically engages the magnetic stir bar 306 to cause the magnetic stir bar 306 to rotate within the cuvette tank 302, thereby stirring the contents of the cuvette tank 302, such as the water sample and/or any chemicals or reagents as disclosed herein.

According to one aspect of the present disclosure, the magnetic motor 304 is positioned adjacent to and laterally alongside the bottom end portion 308 of the cuvette tank 302 such that the magnetic motor 304 readily engages the magnetic stir bar 306 at a minimum or substantially minimum distance, as shown in FIG. 3.

According to one aspect of the present disclosure, the cuvette assembly 300 also comprises a waterproof barrier 310, disposed between the cuvette tank 302 and the magnetic motor 304. Advantageously, the waterproof barrier 310 protects the magnetic motor 304 and any electrical components thereof from spillage and splashing from the cuvette tank 302. Furthermore, by spacing the magnetic motor 304 slightly laterally apart from the cuvette tank 302, access to the cuvette tank 302 can be made easier, for example from cleaning. Additionally, by placing the magnetic stir bar 306 at a lateral side portion of the cuvette tank 302, as shown in FIG. 3, the reliability and efficiency of the stirring can be improved and the likelihood of splashing reduced.

It will be appreciated that, in some aspects of the present disclosure, the various devices disclosed herein can be assembled and used in combination. For example, the liquid test system 100 disclosed herein can be used with either or both of the pumping assembly 200 and the cuvette assembly 300 with magnetic stir bar 306 previously described to provide a test assembly 400. Such examples are described in greater detail below.

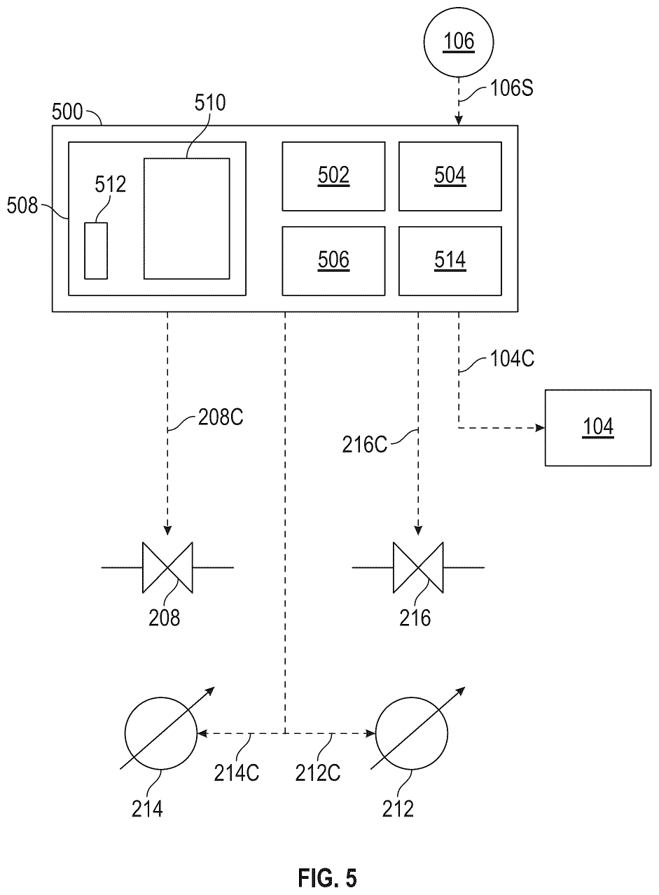

Turning now to FIG. 4, a test assembly 400 is shown according to one aspect of the present disclosure. As shown in FIG. 4, the test assembly 400 comprises the liquid test system 100 and the cuvette assembly 300 in combination. It will be further appreciated that, while not shown in FIG. 4, the components of the test assembly 400, and particularly, the liquid test system 100 can be connected to a pumping assembly 200 as previously described.

As shown in FIG. 4, the test assembly 400 comprises the cuvette 102, the light emitter 104, the light sensor 106, and the reflective surface 116 previously introduced in relation to the liquid test system 100 and previously depicted in FIGS. 1A and 1B. A transparent waterproof barrier 120 can, in some examples, be positioned between the cuvette 102 and the light emitter 104 and light sensor 106 as shown in FIG. 3. This combination of features offers substantially the same advantages as previously discussed in relation to the liquid test system 100.

With continued reference to FIG. 4, the test assembly 400 also includes the magnetic motor 304 and the magnetic stir bar 306 previously introduced in relation to the cuvette assembly 300 shown earlier in FIG. 3. Particularly, the magnetic stir bar 306 can be positioned in the cuvette 102, and more particularly towards a bottom end portion 402 of the cuvette 102.

The magnetic motor 304 is positioned external to the cuvette 102, and is aligned with the magnetic stir bar 306 to allow the magnetic motor 304 to drive the magnetic stir bar 306. For instance, in the example shown in FIG. 4, the magnetic motor 304 is vertically aligned with the magnetic stir bar 306, and positioned to the side of the cuvette 102.

According to one aspect of the present disclosure, such as that shown in FIG. 4, the transparent waterproof barrier 120 can separate the magnetic motor 304 from the cuvette 102, in a similar fashion to the separation of the light emitter 104 and the light sensor 106 from the cuvette 102. For example, the magnetic motor 304 can be mounted to or abut one side of the transparent waterproof barrier 120, with the cuvette 102 on an opposite side of the transparent waterproof barrier 120, as shown in FIG. 4.

As previously described in relation to the cuvette assembly 300 shown in FIG. 3, the magnetic motor 304 can magnetically couple to and drive the magnetic stir bar 306 within the cuvette 102, thereby mixing and homogenizing or partially homogenizing the liquid within the cuvette 102. Thereafter, a light may be emitted from the light emitter 104, passed through the transparent waterproof barrier 120, the cuvette 102, and the homogenized or partly homogenized liquid sample within the cuvette 102 to reflect off the reflective surface 116. The reflected light will pass through the cuvette 102, the homogenized or partly homogenized liquid sample within the cuvette 102, and the transparent waterproof barrier 120, and be collected by the light sensor 106.

The test assembly 400 may also, according to one aspect of the present disclosure, include a pumping assembly such as the pumping assembly 200 disclosed herein, particularly in communication with the cuvette 102 of the water test assembly 400. For example, as shown in FIG. 4, a conduit 206 of the pumping assembly 200 can open into the cuvette 102. In this way, liquids introduced to the pumping assembly 200 from the liquid containers 202 can similarly be introduced to the cuvette 102, in a way substantially similar to that previously discussed. Advantageously, this allows for measured amounts of water and/or reagent to be controllably introduced to the cuvette 102 of the test assembly 400.

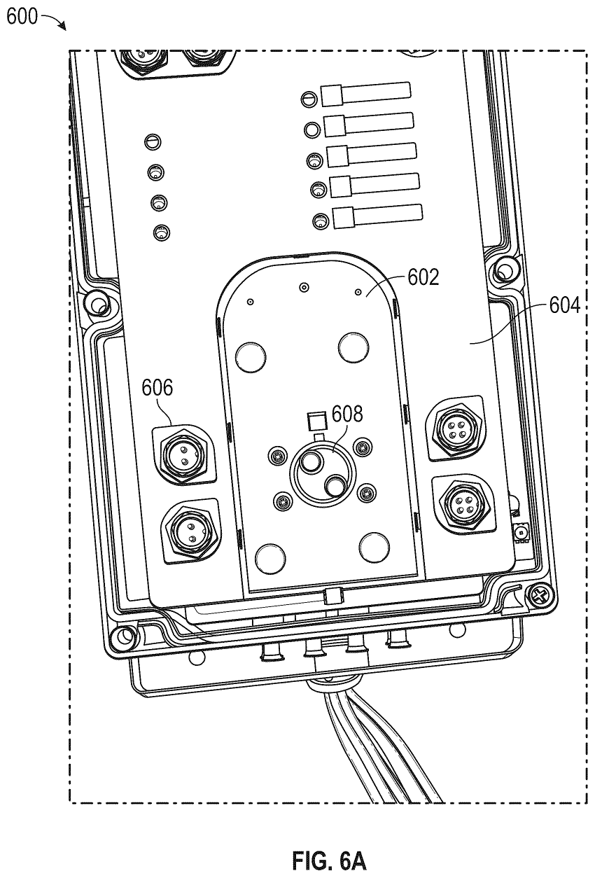

According to one aspect of the present disclosure, the test assembly 400 can also be operatively connected to a controller, such as the controller 500 shown in FIG. 5. The controller 500 includes or may be associated with a processor 502, a computer readable medium 504, a database 506 and an input/output module or control panel 508 having a display 510. The control panel 508 may be accessible through a personal computer or mobile device, or may be a discrete output module or control panel 508 connected directly to the other components of the test assembly 400. An input/output device 512, such as a keyboard, touchscreen, or other user interface, can be provided so that a human operator may input instructions to the controller 500.

It is understood that the controller 500 described herein may be a single controller having the described functionality, or it may include multiple controllers wherein the described functionality is distributed among the multiple controllers.

Various operations, steps or algorithms as described in connection with the controller 500 can be embodied directly in hardware, in a computer program product 514 such as a software module executed by the processor 502, or in a combination of the two. The computer program product 514 can reside in RAM memory, flash memory, ROM memory, EPROM memory, EEPROM memory, registers, hard disk, a removable disk, or any other form of computer-readable medium 504 known in the art. An exemplary computer-readable medium 504 can be coupled to the processor 502 such that the processor 502 can read information from, and write information to, the memory/storage medium. In the alternative, the medium can be integral to the processor. The processor and the medium can reside in an application specific integrated circuit (ASIC). The ASIC can reside in a user terminal. In the alternative, the processor and the medium can reside as discrete components in a user terminal.

The term “processor” as used herein may refer to at least general-purpose or specific-purpose processing devices and/or logic as may be understood by one of skill in the art, including but not limited to a microprocessor, a microcontroller, a state machine, and the like. A processor can also be implemented as a combination of computing devices, e.g., a combination of a DSP and a microprocessor, a plurality of microprocessors, one or more microprocessors in conjunction with a DSP core, or any other such configuration.

According to one aspect of the present disclosure, illustrated in FIG. XX, the controller 500 is configured to receive one or more input signals and to produce and transmit one or more output signals in response to or based in part on the input signals.

For example, if the controller 500 is in communication with the light emitter 104 and the light sensor 106, the controller 500 may transmit a light emitter control signal 104C, which controls the activation and/or the deactivation of the light emitter 104 (for example, instructing a transmission of light and/or termination of the transmission of light).

The controller 500 can also receive one or more light sensor signals 106S transmitted from the light sensor 106 to the controller 500. The one or more light sensor signals 106S can be based on the light received by the light sensor 106, as described above. In such examples, the controller 500 may be configured, for example through the operation of the processor 502 and/or the computer readable medium 504, to interpret the data contained in the one or more light sensor signals 106S to produce one or more characteristic factors, such as wavelength, intensity, transmissivity, and/or absorption of the light received by the light sensor 106. According to one aspect of the present disclosure, these characteristic factors of the one or more light sensor signals 106S can be compared to one or more target values or target signal values, and this comparison may be used to determine or generate results for one or more tests. The generated test results can be displayed on the display 510, and/or transmitted to another device, such as a computer or mobile device.

The controller 500 may also control the admission of liquids into the cuvette 102. In such examples, the controller 500 may be operatively connected to one or more of the liquid valves 208, 216 or the pumps 212, 214 of the pumping assembly 200. In such examples, the controller 500 can transmit liquid valve control signals 208C and airtight valve control signals 216C to the liquid valves 208 and the airtight valve 216 of the pumping assembly 200, respectively, which may open and/or close the liquid valves 208, 216. Similarly, the controller 500 can transmit control signals 212C and 214C to the liquid pump 212 and the air pump 214, respectively, which can activate and/or deactivate the pumps 212, 214.

In such examples, the controller 500 may operate according to a test program to introduce specific and predesignated amounts of liquid into the cuvette 102 through the combined operation of the liquid valves 208, 216, and the pumps 212, 214. Additionally, the controller may flush the 400, for example with air or clean water, before, during, or after a test operation, as required by a test scheme or program. One or more test programs can be stored in the computer readable medium 504, or can be supplied externally, for example, on an external computer or mobile device.

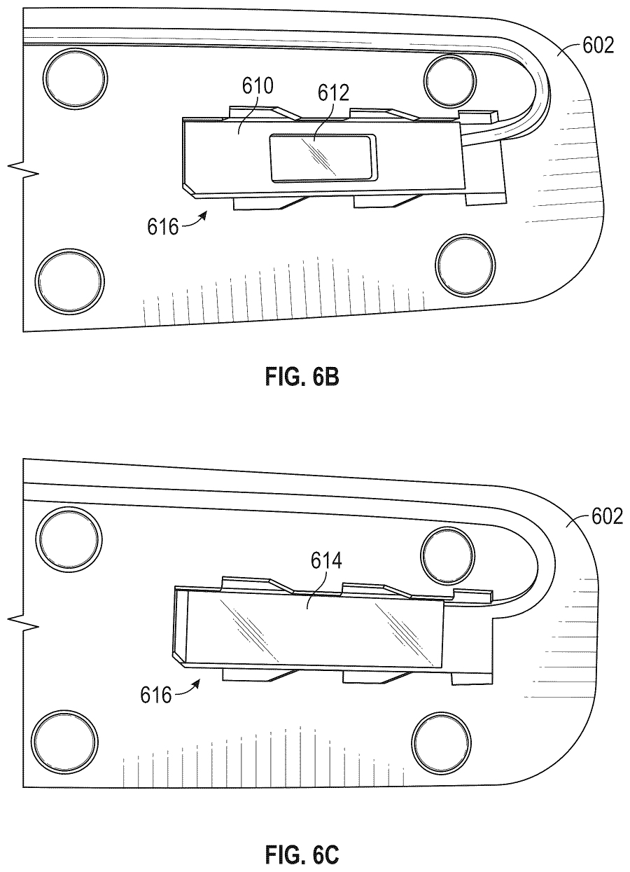

An example test assembly 600 according to one aspect of the present disclosure is shown in FIGS. 6A-6C. As shown in FIG. 6A, the test assembly 600 includes a test assembly body 602 mounted in a fixed housing 604. The fixed housing 604 may, in some examples such as that shown in FIG. 6A, include one or more communication ports 606. The one or more communication ports 606 can receive electrical or data connections, for example, to connect the test assembly 600 to a power source or to a controller, such as the controller 500 described herein. The test assembly 600 can also include a magnetic stirring assembly, 608, which may have substantially the same features as the magnetic stirring assembly described previously in association with them cuvette assembly 300.

FIGS. 6B and 6C show a cutaway of the test assembly body 602. As shown in FIGS. 6B and 6C, the test assembly 600 can include a cuvette assembly 614 set within a cavity in the test assembly body 602. The cuvette assembly 608 can include a cuvette 610, which can be substantially similar to the cuvette 102 previously introduced in relation to the liquid test system 100. A transparent waterproof barrier 612 can admit light into the 610, such that light passes through the cuvette 610 and the contents of the cuvette 610. The transparent waterproof barrier 612 can be substantially similar to the transparent waterproof barrier 120 previously introduced in relation to the liquid test system 100.

The test assembly 600 can also include a reflective surface 614, which as shown in FIG. 6C, can be set within the cavity in the test assembly body 602, such that the cuvette 610 sits between the transparent waterproof barrier 612 and the reflective surface 614. With this arrangement of features, light transmitted through the cuvette 610 and the liquid in the cuvette 610 will reflect off the reflective surface 614 and pass through the cuvette 610 and the contents thereof a second time.

In such examples, a light emitter and a light detector, such as the light emitter 104 and the light sensor 106 previously introduced in relation to the liquid test system 100 may be positioned against the transparent waterproof barrier 612 so that light from the light emitter may pass through the transparent waterproof barrier 612, the cuvette 610, the contents of the cuvette 610, and reflect off the reflective surface 614 as previously described, such that the light detector can receive the reflected light and, in some examples, transmit a response signal to a controller such as the controller 500 described herein.

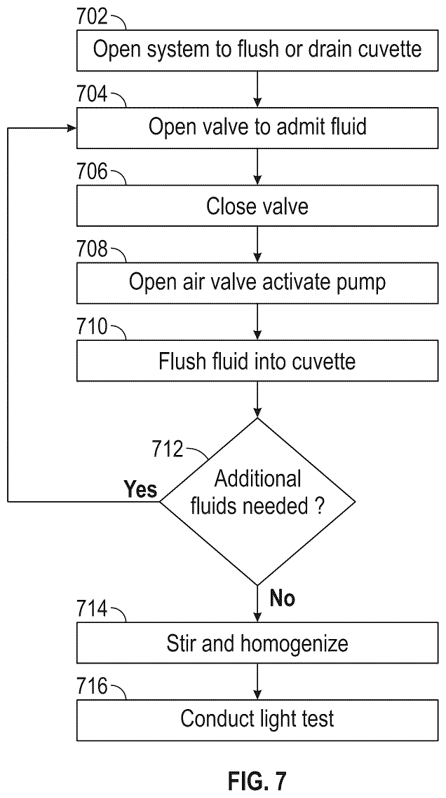

Also disclosed herein are example water test methods using a liquid test system including one or more of the features, systems, or assemblies disclosed herein. An example water test method 700 is shown in FIG. 7, for use with a system comprising a cuvette, such as the cuvette 102 previously introduced, and a pumping assembly such as the pumping assembly 200 previously introduced.

According to one aspect of the present disclosure, as shown in FIG. 7, the cuvette is drained of previously present liquids to ensure that only desired liquids are within the cuvette during the testing, as indicated in draining step 702. According to some aspects of the present disclosure, the cuvette may be drained by opening a draining valve or running a draining pump, but it will be appreciated that various methods may be used to empty the cuvette.

After the cuvette has been emptied of previously present liquids, new liquids are introduced to the cuvette 102 through the pumping assembly 200, as indicated in first liquid introduction step 704. Particularly, a measured volume of a first liquid, such as water from an aquarium, is admitted from a liquid tank of the liquid containers 202 into the manifold 204 through one of the one or more liquid valves 208. According to one example, the predetermined volume of the first liquid may range from 1.0 mL to 2.0 mL, such as 1.0 mL, 1.1 mL, 1.2 mL, 1.3 mL, 1.4 mL, 1.5 mL, 1.6 mL, 1.7 mL, 1.8 mL, 1.9 mL, or 2.0 mL.

After the first liquid has been admitted to the manifold, the first valve is closed off to isolate the first liquid in the manifold 204, as indicated in valve closure step 706. With the liquid isolated in the manifold 204, the airtight valve 216 can be opened, and optionally, the air pump 214 can be activated, to admit (or, if the air pump 214 is activated, to force) the first liquid from the manifold 204 to the cuvette 102, as air displaces the first liquid within the manifold 204, as indicated in steps 708 and 710 of FIG. 7.

With the manifold thus clear of the first liquid, additional liquids may be added. For example, if a user (or in cases where the pumping assembly is operated by a controller, such as the controller 500 previously introduced, a program), determines in additional liquid requirement check step 712, that additional liquids are needed in the cuvette 102, then the previous steps 704 through 710 can be executed again. It will be appreciated that these steps may be executed as needed to add as many liquids, such as water and reagents, to the cuvette 102.

Alternatively, if no additional liquids must be added to the cuvette 102 in order to prepare the test, the liquids within the cuvette 102 can be homogenized or substantially homogenized, as indicated in homogenization step 714. In examples in which the liquid test system 100 and the pumping assembly 200 are used in combination with a magnetic stirring system with a magnetic stir bar such as the magnetic stir bar 306 and a magnetic motor such as the magnetic motor 304 previously introduced, the magnetic stir bar 306 can be actuated within the cuvette 102 to homogenize or substantially homogenize the liquids within the cuvette 102.

With the liquids in the cuvette 102 homogenized, it is then possible to execute one or more light tests, using light emitters and receivers, such as the light emitter 104 and the light sensor 106 previously introduced in relation to the liquid test system 100.

Advantageously, this combination of methods and features improves the precision of the admission of liquids to a cuvette such as the cuvette 102. By clearing the manifold 204 of previously introduced liquids between the introduction of each new liquid such as water or a reagent, and particularly when air pressure is used to ensure that the manifold 204 is substantially free of residual material, and that all introduced material is transferred from the manifold 204 to the cuvette 102.

Furthermore, by preventing liquids from collecting and remaining in the manifold 204, and the valves 208, 216, introduced liquids, and particularly chemical reagents, are prevented from drying and hardening within the pumping assembly 200.

Additionally, in some cases, the reagents may be solutions or suspensions that require periodic agitation to prevent from separating into their discrete constituents. Because the liquid containers 202 which contain the reagents are in communication with an air pump according to some aspects of the present disclosure, the air pump can be used to introduce a small volume of air to agitate or “bubble” the reagent solutions, thereby preventing settling or separation.

### Additional Examples

This written description uses examples to illustrate the various aspects of the disclosed technology, and to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

Example 1. A liquid test system, comprising a cuvette configured to contain a water sample; a reflective surface positioned on a first side of the cuvette a light emitter positioned on a second side of the cuvette opposite to the reflective surface; and a light sensor positioned on a second side of the cuvette opposite to the reflective surface; wherein the light emitter is configured to transmit a light through the cuvette and the water sample, wherein the reflective surface is configured to receive the light transmitted through the cuvette and the water sample and reflect the light through the cuvette and the water sample, and wherein the light sensor is configured to receive the reflected light and to generate a sensor signal based on the reflected light.

Example 2. The liquid test system of any example herein, particularly example 1, further comprising a pumping assembly in communication with the cuvette, the pumping assembly configured to introduce a reagent to the water sample in response to a user input.

Example 3. The liquid test system of any example herein, particularly example 1, further comprising a transparent waterproof barrier disposed between the light emitter and the cuvette.

Example 4. The liquid test system of any example herein, particularly example 3, wherein the transparent waterproof barrier is also disposed between the light sensor and the cuvette.

Example 5. The liquid test system of any example herein, particularly example 1, further comprising a controller in communication with the light sensor and the light emitter, wherein the controller is configured to transmit a light emitter control signal to the light emitter and receive a sensor signal from the light sensor.

Example 6. The liquid test system of any example herein, particularly example 5, wherein the controller includes a processor and a computer readable medium, and wherein the computer readable medium stores one or more test programs, and wherein the controller is configured to compare the sensor signal to one or more target values in the one or more test programs to produce a test result based on the comparison of the sensor signal to the one or more target values.

Example 7. The liquid test system of any example herein, particularly example 1, further comprising a magnetic stir bar disposed within the cuvette and a magnetic motor disposed outside of the cuvette, wherein the magnet is configured to magnetically actuate the magnetic stir bar to stir the liquid sample in the cuvette.

Example 8. The liquid test system of any example herein, particularly example 7, wherein the magnetic motor is vertically aligned with and laterally spaced apart from the magnetic stir bar.

Example 9. A liquid test system, comprising a cuvette configured to contain a water sample; a light emitter positioned adjacent to the cuvette; a light sensor positioned adjacent to the cuvette and alongside the light emitter; and a pumping assembly including a manifold, a liquid pump in communication with the manifold, an airtight valve in communication with the manifold, and one or more liquid containers in communication with the manifold; wherein the light emitter is configured to transmit a light through the cuvette and the water sample, wherein the light sensor is configured to receive the transmitted light and to generate a sensor signal based on the received light, wherein the liquid pump is disposed between the manifold and the cuvette, and configured to introduce liquids from the manifold to the cuvette, wherein the airtight valve is configured to introduce an airflow into the manifold, such that a liquid content of the manifold is expelled from the manifold.

Example 10. The liquid test system of any example herein, particularly example 9, further comprising an air pump in communication with the airtight valve, wherein the airflow is a pressurized airflow.

Example 11. The liquid test system of any example herein, particularly example 9, further comprising one or more liquid valves corresponding to the one or more liquid containers and disposed between the one or more liquid containers and the manifold.

Example 12. The liquid test system of any example herein, particularly example 9, further comprising a controller in communication with the pumping assembly, and configured to control a volume of a liquid introduced to the cuvette through the manifold.

Example 13. A water test method, the method comprising: introducing a liquid sample to a cuvette through a conduit; introducing a reagent to the liquid sample; transmitting light a first time through the liquid sample such that the light reflects off a reflective surface; transmitting the light through the liquid sample a second time such that the light is received by a light sensor.

Example 14. The water test method of any example herein, particularly example 13, further comprising generating a sensor signal based on the reception of the light by the light sensor.

Example 15. The water test method of any example herein, particularly example 14, further comprising comparing the sensor signal to a target signal value to determine a test result based on the comparison of the sensor signal to the target signal value.

Example 16. The water test method of any example herein, particularly example 13, further comprising agitating the liquid sample and the reagent, to produce a homogenized liquid sample.

Example 17. The water test method of any example herein, particularly example 16, wherein the liquid sample and the reagent are agitated by a magnetic stir bar.

Example 18. The water test method of any example herein, particularly example 13, further comprising flushing the cuvette before introducing the liquid sample or the reagent.

Example 19. The water test method of any example herein, particularly example 18, wherein the cuvette is flushed by introducing pressurized air.

Example 20. The water test method of any example herein, particularly example 13, further comprising flushing the conduit after the light has been received by the light sensor.

Thus, although there have been described particular embodiments of the present invention of a new and useful LIQUID TEST SYSTEM it is not intended that such references be construed as limitations upon the scope of this invention except as set forth in the following claims.

What is claimed is:

1. A liquid test system, comprising:
a cuvette configured to contain a water sample;
a reflective surface positioned on a first side of the cuvette;
a light emitter positioned on a second side of the cuvette opposite to the reflective surface; and
a light sensor positioned on a second side of the cuvette opposite to the reflective surface;
wherein the light emitter is configured to transmit a light through the cuvette and the water sample,
wherein the reflective surface is configured to receive the light transmitted through the cuvette and the water sample and reflect the light through the cuvette and the water sample, and
wherein the light sensor is configured to receive the reflected light and to generate a sensor signal based on the reflected light.

2. The liquid test system of claim 1, further comprising a pumping assembly in communication with the cuvette, the pumping assembly configured to introduce a reagent to the water sample in response to a user input.

3. The liquid test system of claim 1, further comprising a transparent waterproof barrier disposed between the light emitter and the cuvette.

4. The liquid test system of claim 3, wherein the transparent waterproof barrier is also disposed between the light sensor and the cuvette.

5. The liquid test system of claim 1, further comprising a controller in communication with the light sensor and the light emitter, wherein the controller is configured to transmit a light emitter control signal to the light emitter and receive a sensor signal from the light sensor.

6. The liquid test system of claim 5, wherein the controller includes a processor and a computer readable medium, and wherein the computer readable medium stores one or more test programs, and wherein the controller is configured to compare the sensor signal to one or more target values in the one or more test programs to produce a test result based on the comparison of the sensor signal to the one or more target values.

7. The liquid test system of claim 1, further comprising a magnetic stir bar disposed within the cuvette and a magnetic motor disposed outside of the cuvette, wherein the magnet is configured to magnetically actuate the magnetic stir bar to stir the liquid sample in the cuvette.

8. The liquid test system of claim 7, wherein the magnetic motor is vertically aligned with and laterally spaced apart from the magnetic stir bar.

9. A liquid test system, comprising:
a cuvette configured to contain a water sample;
a light emitter positioned adjacent to the cuvette;
a light sensor positioned adjacent to the cuvette and alongside the light emitter; and
a pumping assembly including a manifold, a liquid pump in communication with the manifold, an airtight valve in communication with the manifold, and one or more liquid containers in communication with the manifold;
wherein the light emitter is configured to transmit a light through the cuvette and the water sample,
wherein the light sensor is configured to receive the transmitted light and to generate a sensor signal based on the received light,
wherein the liquid pump is disposed between the manifold and the cuvette, and configured to introduce liquids from the manifold to the cuvette,
wherein the airtight valve is configured to introduce an airflow into the manifold, such that a liquid content of the manifold is expelled from the manifold.

10. The liquid test system of claim 9, further comprising an air pump in communication with the airtight valve, wherein the airflow is a pressurized airflow.

11. The liquid test system of claim 9, further comprising one or more liquid valves corresponding to the one or more liquid containers and disposed between the one or more liquid containers and the manifold.

12. The liquid test system of claim 9, further comprising a controller in communication with the pumping assembly, and configured to control a volume of a liquid introduced to the cuvette through the manifold.

13. A water test method, the method comprising:
introducing a liquid sample to a cuvette through a conduit;
introducing a reagent to the liquid sample;
transmitting light a first time through the liquid sample such that the light reflects off a reflective surface; and
transmitting the light through the liquid sample a second time such that the light is received by a light sensor.

14. The water test method of claim 13, further comprising generating a sensor signal based on the reception of the light by the light sensor.

15. The water test method of claim 14, further comprising comparing the sensor signal to a target signal value to determine a test result based on the comparison of the sensor signal to the target signal value.

16. The water test method of claim 13, further comprising agitating the liquid sample and the reagent, to produce a homogenized liquid sample.

17. The water test method of claim 16, wherein the liquid sample and the reagent are agitated by a magnetic stir bar.

18. The water test method of claim 13, further comprising flushing the cuvette before introducing the liquid sample or the reagent.

19. The water test method of claim 18, wherein the cuvette is flushed by introducing pressurized air.

20. The water test method of claim 13, further comprising flushing the conduit after the light has been received by the light sensor.
